(19) United States
(12) Patent Application Publication
(10) Pub. No.: US 2010/0061887 A1
(43) Pub. Date: Mar. 11, 2010

(54) Ultraviolet Sterilizer for Cosmetic Applicators

(76) Inventors: Jacquelyn C. Harper, Everett, MA (US); Douglas P. Burum, Acton, MA (US)

Correspondence Address: Vern Maine & Associates, 100 MAIN STREET, P O BOX 3445, NASHUA, NH, 03061-3445, US

(21) Appl. No.: 12/547,448

(22) Filed: Aug. 25, 2009

Related U.S. Application Data
(60) Provisional application No. 61/093,426, filed on Sep. 1, 2008.

(51) Int. Cl. A61L 2/10; B01J 19/10; B01J 19/12

(52) U.S. Cl. 422/24; 422/186.3; 422/114; 422/128; 422/119

(57) ABSTRACT

An ultraviolet sterilizer and method of use thereof are disclosed for rapidly and conveniently sterilizing the end of a tubular cosmetic applicator, such as an eyeliner pencil or a lipstick. A hole in the sterilizer allows an end of the cosmetic applicator to be inserted for sterilization. A hole blocking mechanism, such as an iris, can be included, and can be automatically activated by a sensor when an applicator is inserted through the hole. Some embodiments are battery powered and portable, and some of these are wearable by a strap or attachable to a belt. Various embodiments include a brush bristle manipulating structure and/or an ultrasonic source to agitate and expose contaminants during sterilization. Embodiments are able to kill 99 percent of accessible bacteria after a 10 second exposure. Some embodiments further include a drawer or other access mechanism that allows one or more complete applicators to be simultaneously sterilized.

### CROSS-REFERENCES TO RELATED APPLICATIONS

This application is entitled to the benefit of U.S. Provisional Patent Application No. 61/093,426 entitled “Ultraviolet Sterilizer for Cosmetic Applicators,” filed Sep. 1, 2008, herein incorporated by reference in its entirety for all purposes.

### FIELD OF THE INVENTION

The invention generally relates to cosmetics and cosmetic applicators, and more specifically to devices for sterilizing cosmetic applicators.

### BACKGROUND OF THE INVENTION

The retail sale of facial cosmetics, including lipstick, lip pencils, eyeliner pencils, mascara, foundation sticks, etc, is a huge and highly competitive industry, in which success depends heavily on the skillful and efficient demonstration of these products to customers. Typically, a customer will stop at a cosmetics counter in a department store or cosmetics boutique and ask for product advice from a professional makeup artist. After asking a few questions and providing some initial information, the makeup artist will often demonstrate products by applying samples to the customer's face. Often, the makeup artist will perform a complete makeup application on the customer, so as to demonstrate what can be achieved by applying a customized selection of products specifically chosen for the customer. While time consuming, this approach can lead to significant increases in sales.

Because customers vary widely in skin type, coloration, facial features, and makeup preferences, it is necessary to see the effect of cosmetic products on each individual customer before a determination can be made as to which products will be most appropriate. Viewing photographs of products applied to models, or even seeing products worn by a makeup artist or other individuals, is far from adequate. Test applicators, or “testers,” are typically provided at each cosmetic sales station for use by customers who wish to sample products and by makeup artists when demonstrating products to customers. The use of these testers allows merchandise intended for sale to remain undisturbed until it is purchased.

The repeated use of facial cosmetic testers on multiple customers requires that the testers be sterilized between uses, so as to avoid transferring bacteria from one customer to another. A variety of sterilization methods is used, according to the nature of each cosmetic applicator. Chemically and physically resilient applicators such as makeup brushes are typically rinsed using a disinfectant solution. Lipsticks, on the other hand, are too fragile for this method, and are typically “shaved” by rubbing them against a paper towel so as to remove the outer layer of lipstick along with any bacteria that is present.

The cosmetic industry is fundamentally a volume business, in that it depends heavily on minimizing costs while obtaining the maximum amount of sales from each hour worked by each makeup artist. It is not uncommon for a facial cosmetics counter to experience prolonged periods during which a steady stream of customers ask for assistance. Under these circumstances, the time devoted by makeup artists to sterilizing cosmetic applicators between uses can be very costly, since an inability to serve the next customer in a timely fashion can lead to lost sales and reduced profits. In addition, the consumption of large quantities of anti-bacterial solution used for sterilizing cosmetic applicators is costly, as well as harmful to the environment. Also, the cosmetics themselves are costly, and therefore the accelerated consumption of testers due to scraping and other sterilization methods is a further drain on profits.

Ultraviolet sterilizers, sometimes called “UV” sterilizers, have long been used in hospitals and other medical environments for sterilizing surgical instruments. Similar units have been proposed for killing bacteria on cosmetic applicators. Typically, a makeup artist is required to leave his or her station and bring any testers that need sterilization to the UV sterilizer. The sterilizer is opened, one or more testers are inserted, the sterilizer is closed, the ultraviolet lamps are activated for a period sufficient to sterilize the testers, the sterilizer is opened again, the testers are removed, and the makeup artist finally returns to his or her station. While this approach offers the advantages of killing bacteria without damaging or needlessly consuming cosmetic testers, and without the use of anti-bacterial solutions, this approach saves little if any time as compared to conventional methods, and has not been widely put into practice.

### SUMMARY OF THE INVENTION

An ultraviolet (“UV”) cosmetic sterilizer and method of use are claimed that provide for convenient and rapid sterilization of cosmetic applicators as they are used, without requiring a cosmetic makeup artist using the invention to leave his or her station or to otherwise interrupt the flow of the sales process, and without the need to “shave” or otherwise waste valuable product. The UV cosmetic sterilizer also eliminates the need to sterilize cosmetics using tissues and/or other cleaning media, and also eliminates the need to use costly sterilizing solutions, some of which are toxic and/or harmful to the environment. The UV sterilizer offers similar advantages in other environments where cosmetic applicators may be shared. Examples include performers applying makeup in preparation for a performance and models preparing for a fashion show.

The UV cosmetic sterilizer takes advantage of the fact that most facial cosmetic applicators are long and tubular in shape. Examples include makeup brushes, lipsticks, lip pencils, mascara wands, foundation sticks, eyeliner pencils and many other cosmetic applicators. A hole is conveniently located in a wall of the sterilizer, so as to allow the distal end of a cosmetic applicator to be inserted through the hole and sterilized, without the time consuming steps of opening and inserting the applicator before use, and re-opening and removing the applicator after use. The sterilization process typically requires only a few seconds.

In preferred embodiments a mechanism such as an iris is included so as to allow the size of the hole to be dynamically adjusted to the diameter of each makeup applicator to be sterilized. In some preferred embodiments, the sterilizer is portable, can be suspended from a strap and/or attached to a belt, and can be operated by standard and/or rechargeable batteries. Other embodiments are “desktop” models, and some of these include drawers and/or other provisions for sterilizing non-tubular makeup applicators and/or for sterilizing a plurality of applicators simultaneously. Some embodiments include brush manipulating structures for manipulating and spreading the bristles of brushes during sterilization, while other embodiments apply ultrasonic sound in concert with UV light, so as to drive contaminants away from brushes and other applicators and into the surrounding air where they can be more easily killed by the UV radiation.

One general aspect of the present invention is an ultraviolet sterilizer for sterilizing a cosmetic applicator. The ultraviolet sterilizer includes the following elements:

a housing with an interior;

at least one ultraviolet lamp installed and enclosed within the interior of the housing;

a power source connected to the at least one ultraviolet lamp and able to cause the at least one ultraviolet lamp to emit sterilizing ultraviolet light; and

a hole in the housing.

The above elements are proportioned and configured so as to enable a distal end of the cosmetic applicator to be inserted through the hole and into the interior of the housing while a proximal end of the cosmetic applicator remains outside of the housing, thereby enabling the distal end of the cosmetic applicator to be sterilized by sterilizing ultraviolet light emitted by the at least one ultraviolet lamp.

In some preferred embodiments the power source includes batteries, and in some of these embodiments the batteries are rechargeable. In other of these embodiments the ultraviolet sterilizer is able to cause the at least one ultraviolet lamp to emit sterilizing ultraviolet light for an aggregate duration of at least 10 minutes without need to replenish the batteries.

In other preferred embodiments the power source is able to derive power from a conventional electrical wall outlet.

In various preferred embodiments the power source is able to cause the at least one ultraviolet lamp to emit sterilizing ultraviolet light of sufficient intensity to kill 99 percent of accessible bacteria located within the interior of the housing after a ten second exposure, the accessible bacteria being those bacteria that are directly exposed to the sterilizing ultraviolet light when the sterilizing light is emitted from the at least one ultraviolet lamp.

In preferred embodiments, the ultraviolet sterilizer is portable. In some of these embodiments, the ultraviolet sterilizer further includes a carrying strap and/or a belt attachment mechanism that enables attachment of the housing to a belt worn by an individual.

In various preferred embodiments the housing further includes an access mechanism configured so as to enable a cosmetic applicator to be fully inserted into the housing, such that the cosmetic applicator can be sterilized by sterilizing ultraviolet light emitted by the at least one ultraviolet lamp while the cosmetic applicator is completely contained within the housing. In some of these embodiments the access mechanism is configured so as to enable a plurality of cosmetic applicators to be fully inserted into the housing, thereby enabling the plurality of cosmetic applicators to be simultaneously sterilized by sterilizing ultraviolet light emitted by the at least one ultraviolet lamp. And in some of these embodiments the access mechanism is a drawer.

In preferred embodiments the ultraviolet sterilizer further includes a hole-blocking mechanism that is able to substantially prevent ultraviolet light from passing through the hole while a distal end of a cosmetic applicator is inserted through the hole and into the housing. In some of these embodiments the hole-blocking mechanism includes an iris that is able to close around the distal end of the cosmetic applicator. And some of these embodiments further include a sensor and an automatic iris closing mechanism, the sensor being able to detect insertion of the distal end of the cosmetic applicator into the hole, and upon such detection being able to activate the automatic iris closing mechanism so as to cause the iris to close around the distal end of the cosmetic applicator

Various preferred embodiments further include a brush manipulating structure configured and located with the housing so as to manipulate the configuration of the hairs of a brush and thereby increase exposure of the hairs to ultraviolet, the manipulation being caused by movement of the brush manipulating structure relative to the brush and/or movement of the brush relative to the brush manipulating structure.

Some preferred embodiments further include an ultrasonic source attached to the housing and connected to the power source, the ultrasonic source being and able to emit ultrasonic sound into the interior of the housing.

Certain preferred embodiments include a sensor that is able to detect insertion of a cosmetic applicator into the interior of the housing. Some embodiments include a timer configured so as to activate the at least one ultraviolet lamp and then deactivate the at least one ultraviolet lamp after a sterilizing period of time has elapsed. And various embodiments include an alerting system that is able to alert a user when a sterilizing period has been completed and the at least one ultraviolet lamp has been deactivated. In some of these embodiments, the alerting system includes a light that is illuminated when the sterilizing period has been completed and/or a sound producing device that emits an audible sound when the sterilizing period has been completed.

Another general aspect of the invention is a method for sterilizing a cosmetic applicator. The method comprises the following steps:

providing an ultraviolet sterilizer that includes a housing with an interior within which at least one ultraviolet lamp is installed and enclosed, a power source connected to the at least one ultraviolet lamp and able to cause the at least one ultraviolet lamp to emit sterilizing ultraviolet light, and a hole in the housing through which a distal end of the cosmetic applicator can be inserted into the interior of the housing;

inserting the distal end of the cosmetic applicator through the hole and into the interior of the housing; activating the power source, so as to cause the at least one ultraviolet lamp to emit sterilizing ultraviolet light;

allowing the sterilizing ultraviolet light to impinge on the distal end of the cosmetic applicator until the amount of ultraviolet light exposure is sufficient to sterilize the distal end of the cosmetic applicator; and

removing the distal end of the cosmetic applicator from the hole.

### BRIEF DESCRIPTION OF THE DRAWINGS

FIG. 1 is a cutaway drawing of a preferred embodiment that is cylindrical in shape, includes four ultraviolet lamps, and is battery powered;

FIG. 2 is a cutaway drawing of the embodiment of FIG. 1, including a top with a hole and an eyeliner pencil inserted through the hole;

FIG. 3 is a cutaway drawing of an embodiment similar to FIG. 2, but including a brush manipulating structure against which the hairs of a brush can be manipulated;

FIG. 4 is a cutaway drawing of an embodiment similar to FIG. 2, but including an ultrasonic source that can apply ultrasonic sound to a cosmetic applicator while it is being sterilized;

FIG. 5A is a side cross sectional view of an embodiment similar to FIG. 1, but including an iris hole blocking mechanism and an optical sensor that senses the presence of a cosmetic applicator;

FIG. 5B is a top view of the embodiment of FIG. 5A configured in preparation for insertion of a cosmetic applicator;

FIG. 5C is a side cross sectional view of the embodiment of FIG. 5A with a cosmetic applicator inserted through the hole and sensed by the sensor;

FIG. 5D is a top view of the embodiment of FIG. 5C with the iris hole blocking mechanism closed around the cosmetic applicator in the hole;

FIG. 6 is a perspective view of a cosmetic makeup artist wearing a portable embodiment of the present invention on a shoulder strap.

FIG. 7A is a perspective view of a desktop embodiment of the present invention that is powered from a conventional wall outlet and includes a drawer that can accept a plurality of cosmetic applicators;

FIG. 7B is a front view of the embodiment of FIG. 7A showing the positioning of the ultraviolet lamps within the sterilizer;

FIG. 7C is a perspective view of the embodiment of FIG. 7A showing an eyeliner pencil inserted through the hole; and

FIG. 7D is a perspective view of the embodiment of FIG. 7A configured with the drawer open and a plurality of cosmetic applicators contained therein.

### DETAILED DESCRIPTION OF PREFERRED EMBODIMENTS

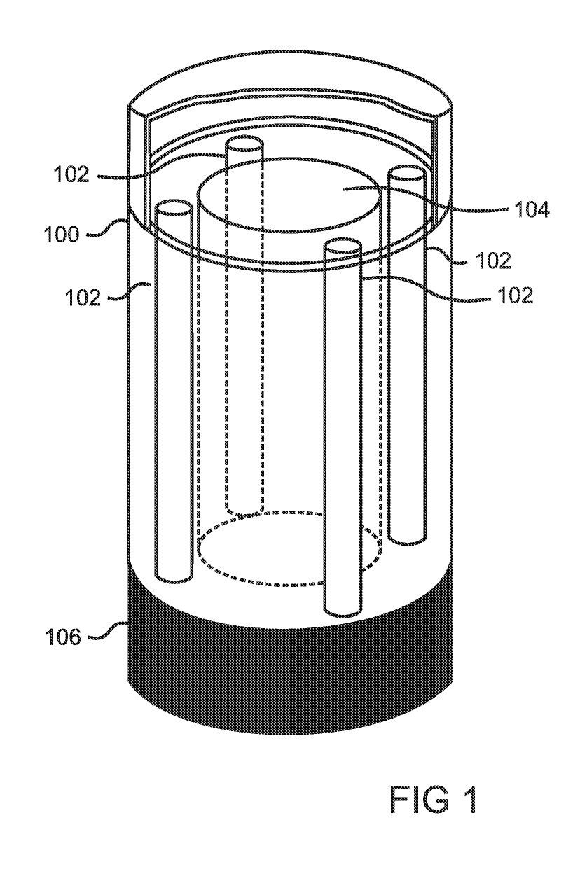

With reference to FIG. 1, the present invention includes a housing 100 with at least one ultraviolet (“UV”) lamp 102 installed in its interior. In the embodiment of FIG. 1, there are four such lamps 102 installed, so as to provide irradiation of an inner section 104 of the interior with UV light from substantially all directions. The UV lamps are powered by batteries contained in a power section 106 located at the bottom of the housing 100.

In preferred embodiments, the UV lamps 102 emit short range, so-called “UV-C” light at a wave length of substantially 254 nm, which is deadly to bacteria, viruses, and other micro-organisms. UV-C light also generates ozone, which is deadly to micro-organisms and thereby increases the effectiveness of the ultraviolet sterilizer. It will be clear to someone of average skill in the art that even though ozone generated by the UV lamps 102 will be deadly to micro-organisms located within the housing 100, ozone will not be generated by the lamps in sufficient quantity to measurably affect the health quality of the surrounding air once the ozone escapes from the housing 100.

So as to kill 99 percent of bacteria located in the interior of the housing 100, an exposure is required of approximately 8500 micro-Watt seconds per square centimeter. A typical, commercially available 4.5 Watt germicidal UV bulb (model G4T5) operating at 29 Volts with a length of five inches produces approximately 3300 micro-Watts of UV-C intensity per square centimeter at a distance of one inch from the bulb. At the same time, the bulb generates approximately 100 micro-grams of ozone per second. A preferred embodiment such as the one illustrated in FIG. 1 that uses four such bulbs 102 will consume 18 watts, and will generate sufficient UV intensity to kill all bacteria within one inch of the bulbs 102 in less than a second. If the ultraviolet sterilizer is used 50 times per day, the aggregate usage will only be about one minute. Even allowing for losses in a ballast, voltage converter, and other components, the energy stored in two typical NiMH AA batteries (about 5 Watt-hours) will be sufficient to operate the device continuously for more than 10 minutes.

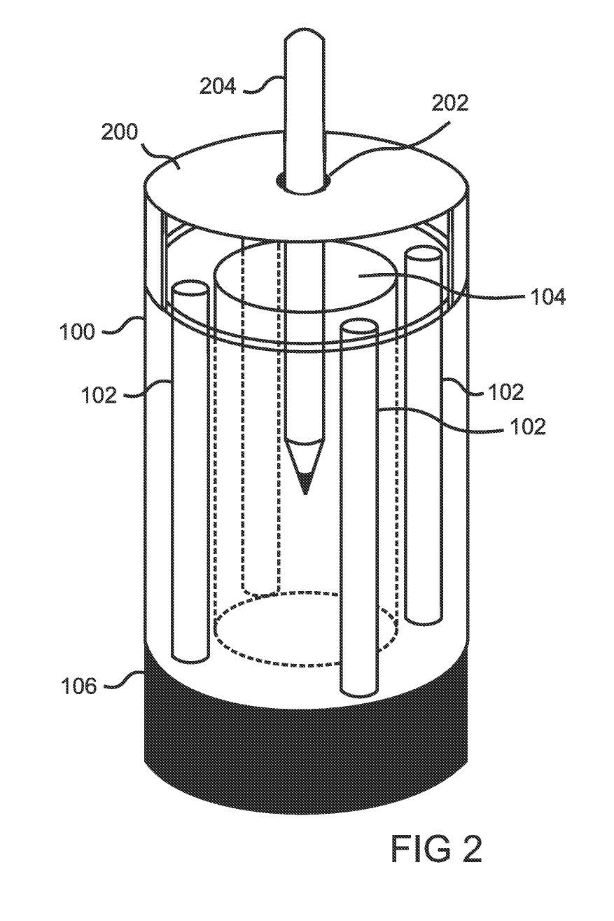

FIG. 2 further illustrates the embodiment of FIG. 1, showing the top 200 of the container 100 with a hole 202 substantially in its center that allows a tubular cosmetic applicator such as the lip liner pencil 204 shown in the figure to be conveniently inserted into the inner section 104 of the interior of the ultraviolet sterilizer without the need to open any covers, drawers, etc.

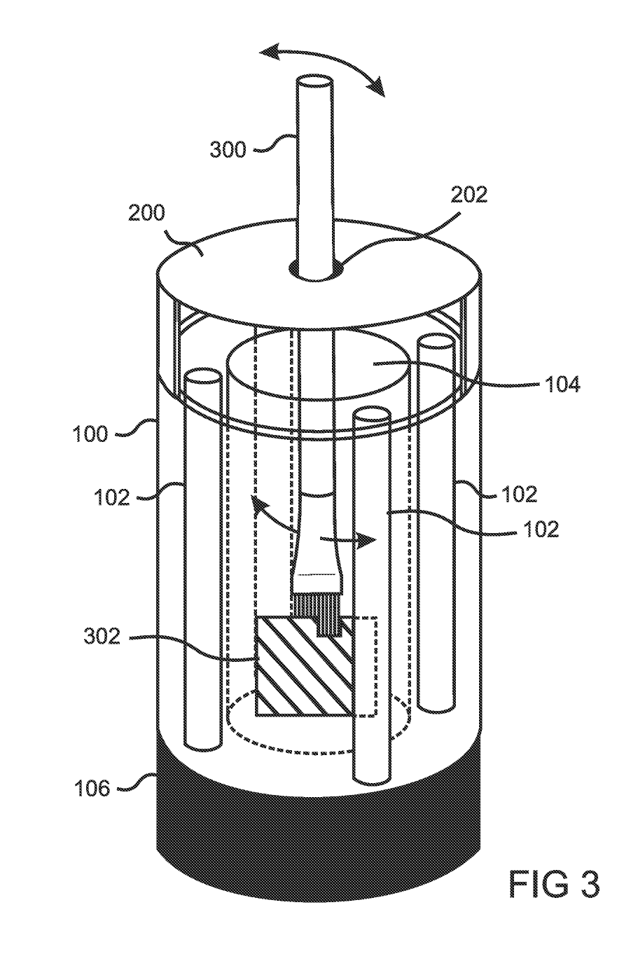

FIG. 3 illustrated an embodiment similar to the embodiment of FIG. 1. A cosmetic brush 300 has been inserted through the hole 202 in the top 200 and into the inner section 104 of the interior of the ultraviolet sterilizer. In this embodiment, a brush manipulating structure 302 is included near the bottom of the inner section 104 of the interior. By moving the tip of the brush 300 against the brush manipulating structure 302 while the sterilizing lamps are illuminated the bristles of the brush are temporarily separated from each other so as to allow the sterilizing light and ozone to more efficiently reach the areas between the bristles.

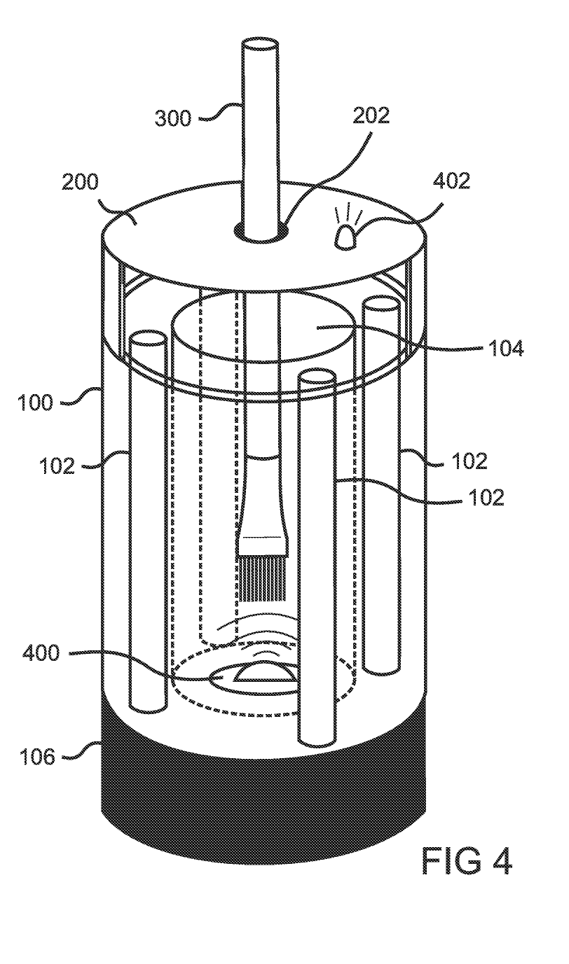

FIG. 4 illustrates an embodiment similar to the embodiment of FIG. 3, except that an ultrasonic source 400 is included at the bottom of the inner section 104 of the interior. The ultrasonic source 400 is able to apply ultrasonic sound waves to the bristles at the tip of the brush 300, so as to loosen any contaminants contained within the bristles and cause them to emerge from the brush so as to be exposed to the sterilizing ultraviolet light and ozone within the inner section 104 of the interior. Similarly, the ultrasonic sound waves are able to loosen contaminants from any surface of any cosmetic applicator, thereby causing them to enter the surrounding air where they can be more easily sterilized by ultraviolet light and ozone. The embodiment of FIG. 4 also includes a sensor (not shown) that detects insertion of a cosmetic applicator into the sterilizer, and a timer (not shown) that activates the ultraviolet lamps 102 for a period sufficiently long to sterilize the cosmetic applicator. At the end of this process a light 402 mounted on the exterior of the housing 100 is illuminated so as to alert a user that it is safe to remove and reuse the cosmetic applicator.

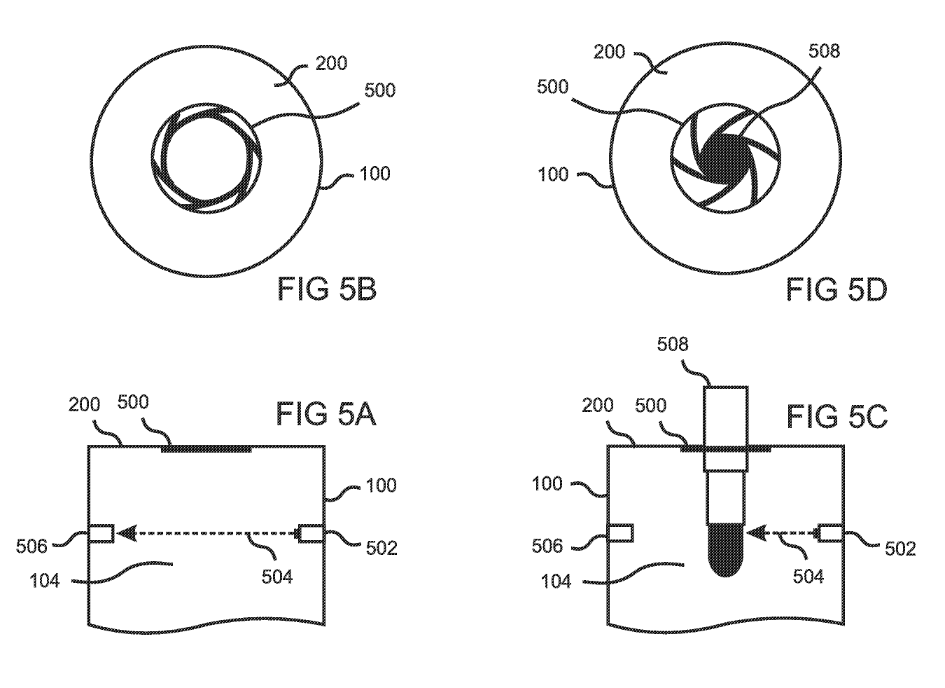

FIG. 5A is a cross section illustration of the top of a preferred embodiment similar to the embodiment of FIG. 1 that includes an iris 500 installed within the hole 202. The iris is normally open so as to allow tubular cosmetic applicators of various diameters to be inserted through the iris and into the inner section 104 of the interior. A diode LASER 502 emits a beam of light 504 across the inner section 104 of the interior below the iris, and the beam of light 504 is detected by a photo-diode 506 on the other side of the interior. When a cosmetic applicator is inserted through the iris 500, the beam of light 504 is interrupted, causing the iris 500 to be automatically closed. FIG. 5B is a top view of the embodiment of FIG. 5A, illustrated with the iris 500 in an open configuration. FIG. 5C is a cross section illustration of the embodiment of FIG. 5A showing a tubular cosmetic applicator 508 inserted through the iris into the inner section 104 of the interior of the ultraviolet sterilizer, such that the cosmetic applicator 508 blocks the beam of light 504, and FIG. 5D is a top view of the embodiment of FIG. 5C showing the iris 500 closed around the tubular cosmetic applicator 508.

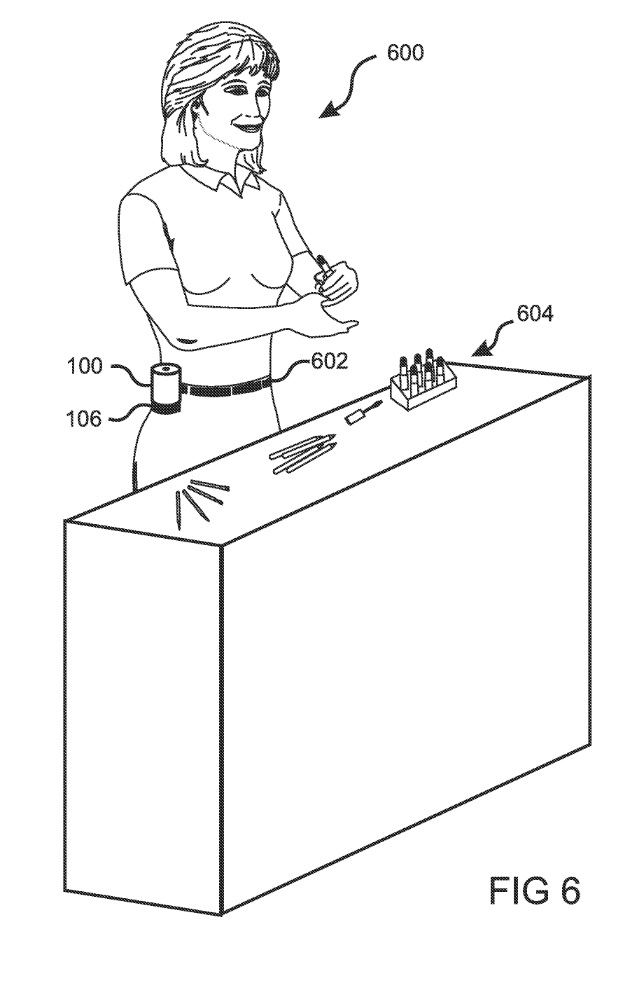

FIG. 6 is a perspective view of a makeup artist 600 wearing the embodiment of FIG. 5A attached to a belt 602 worn by the makeup artist 600. In similar embodiments the ultraviolet sterilizer can be worn using a strap 602 worn around the neck or over a shoulder of the makeup artist 600. Attachment of the ultraviolet sterilizer to the makeup artist 602 allows the makeup artist 602 to have both hands free to show and demonstrate cosmetics 604, and allows tubular cosmetic applicators 604 to be inserted into the ultraviolet sterilizer for nearly instant sterilization, without any need for the makeup artist 600 to leave her post or otherwise interrupt her demonstration and sales activities in any way.

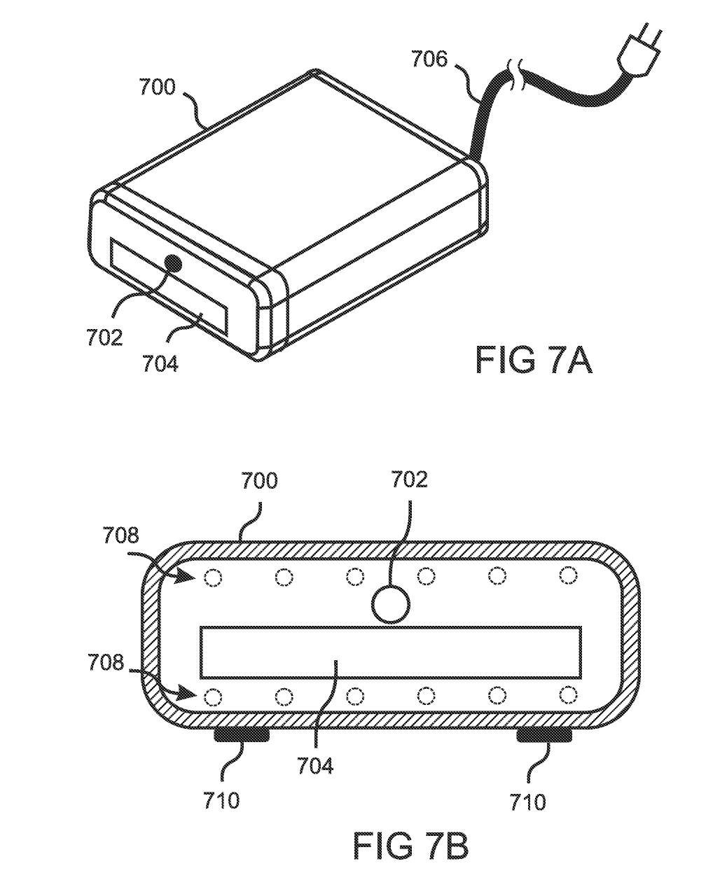

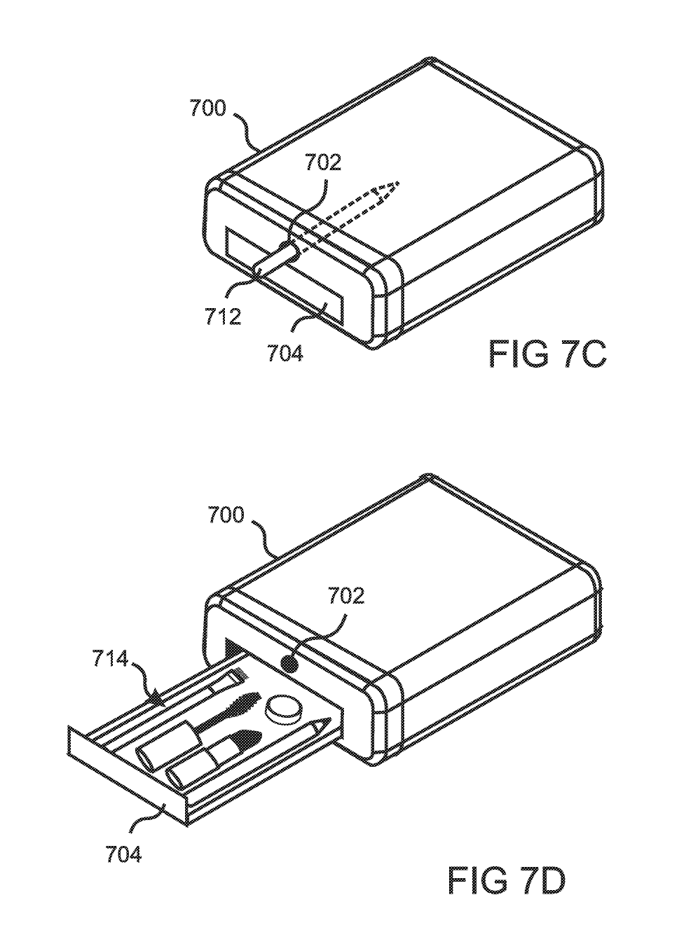

FIG. 7A is a perspective view of a table-top embodiment of the ultraviolet sterilizer. This embodiment includes a rectangular housing 700 with an insertion hole 702 for convenient insertion of the end of an individual, tubular cosmetic applicator as well as a drawer 704 for simultaneous insertion of one or more applicators. Cosmetic applicators inserted into the ultraviolet sterilizer using the drawer are sterilized over their entire length, but usage of the drawer requires the additional steps of opening the drawer both before and after use. By including both an insertion hole 702 and a drawer, this embodiment is applicable for both “on the fly” sterilization of tubular cosmetic applicators during a cosmetic demonstration, and for simultaneous sterilization of a plurality of cosmetic applicators of all shapes in-between cosmetic demonstrations. When a sterilizing period is completed, a sound is emitted by a small speaker (not shown) located inside of the housing 700.

FIG. 7B is a cross-sectional view of the embodiment of FIG. 7A, showing the arrangement of ultra-violet lamps 708 surrounding the sterilization region within the ultraviolet sterilizer. Note that the drawer 704 in this embodiment is constructed from a material that is transparent to the UV-C light emitted by the ultra-violet lamps 708. Non-slip feet 710 are attached to the bottom of the housing 700 to provide stability to the ultraviolet sterilizer while in use.

FIG. 7C is a perspective view of the embodiment of FIG. 7A showing a lip-liner pencil 712 inserted into the insertion hole 702, and FIG. 7D is a perspective view of the embodiment of FIG. 7A showing the drawer 704 open and with an assortment of cosmetic applicators 714 contained in the drawer. Note that not all of the cosmetic applicators 714 in the drawer 704 are tubular.

Other modifications and implementations will occur to those skilled in the art without departing from the spirit and the scope of the invention as claimed. Accordingly, the above description is not intended to limit the invention except as indicated in the following claims.

What is claimed is:

1. An ultraviolet sterilizer for sterilizing a cosmetic applicator, the ultraviolet sterilizer comprising:
a housing with an interior;
at least one ultraviolet lamp installed and enclosed within the interior of the housing;
a power source connected to the at least one ultraviolet lamp and able to cause the at least one ultraviolet lamp to emit sterilizing ultraviolet light; and
a hole in the housing;
the above elements being arranged and configured so as to enable a distal end of the cosmetic applicator to be inserted through the hole and into the interior of the housing while a proximal end of the cosmetic applicator remains outside of the housing, thereby enabling the distal end of the cosmetic applicator to be sterilized by sterilizing ultraviolet light emitted by the at least one ultraviolet lamp.

2. The ultraviolet sterilizer of claim 1, wherein the power source includes batteries

3. The ultraviolet sterilizer of claim 2, wherein the batteries are rechargeable.

4. The ultraviolet sterilizer of claim 2, wherein the ultraviolet sterilizer is able to cause the at least one ultraviolet lamp to emit sterilizing ultraviolet light for an aggregate duration of at least 10 minutes without need to replenish the batteries.

4. The ultraviolet sterilizer of claim 1, wherein the power source is able to derive power from a conventional electrical wall outlet.

5. The ultraviolet sterilizer of claim 1, wherein the power source is able to cause the at least one ultraviolet lamp to emit sterilizing ultraviolet light of sufficient intensity to kill 99 percent of accessible bacteria located within the interior of the housing after a ten second exposure, the accessible bacteria being those bacteria that are directly exposed to the sterilizing ultraviolet light when the sterilizing light is emitted from the at least one ultraviolet lamp.

6. The ultraviolet sterilizer of claim 1, wherein the ultraviolet sterilizer is portable.

7. The ultraviolet sterilizer of claim 6, further comprising at least one of:
a carrying strap; and
a belt attachment mechanism that enables attachment of the housing to a belt worn by an individual.

8. The ultraviolet sterilizer of claim 1, further comprising an access mechanism configured so as to enable a cosmetic applicator to be fully inserted into the housing, such that the cosmetic applicator can be sterilized by sterilizing ultraviolet light emitted by the at least one ultraviolet lamp while the cosmetic applicator is completely contained within the housing.

9. The ultraviolet sterilizer of claim 8, wherein the access mechanism is configured so as to enable a plurality of cosmetic applicators to be fully inserted into the housing, thereby enabling the plurality of cosmetic applicators to be simultaneously sterilized by sterilizing ultraviolet light emitted by the at least one ultraviolet lamp.

10. The ultraviolet sterilizer of claim 8, wherein the access mechanism is a drawer.

11. The ultraviolet sterilizer of claim 1, further comprising a hole-blocking mechanism that is able to substantially prevent ultraviolet light from passing through the hole while a distal end of a cosmetic applicator is inserted through the hole and into the housing.

12. The ultraviolet sterilizer of claim 11, wherein the hole-blocking mechanism includes an iris that is able to close around the distal end of the cosmetic applicator.

13. The ultraviolet sterilizer of claim 12, further comprising a sensor and an automatic iris closing mechanism, the sensor being able to detect insertion of the distal end of the cosmetic applicator into the hole, and upon such detection being able to activate the automatic iris closing mechanism so as to cause the iris to close around the distal end of the cosmetic applicator

14. The ultraviolet sterilizer of claim 1, further comprising a brush manipulating structure configured and located with the housing so as to manipulate the configuration of hairs of a brush and thereby increase exposure of the hairs to ultraviolet light, the manipulation being caused by at least one of:
movement of the brush manipulating structure relative to the brush; and
movement of the brush relative to the brush manipulating structure.

15. The ultraviolet sterilizer of claim 1, further comprising an ultrasonic source attached to the housing and connected to the power source, the ultrasonic source being and able to emit ultrasonic sound into the interior of the housing.

16. The ultraviolet sterilizer of claim 1, further comprising a sensor that is able to detect insertion of a cosmetic applicator into the interior of the housing.

17. The ultraviolet sterilizer of claim 1, further comprising a timer configured so as to activate the at least one ultraviolet lamp and then deactivate the at least one ultraviolet lamp after a sterilizing period of time has elapsed.

18. The ultraviolet sterilizer of claim 1, further comprising an alerting system that is able to alert a user when a sterilizing period has been completed and the at least one ultraviolet lamp has been deactivated.

19. The ultraviolet sterilizer of claim 18, wherein the alerting system includes at least one of:
a light that is illuminated when the sterilizing period has been completed; and
a sound producing device that emits an audible sound when the sterilizing period has been completed.

20. A method for sterilizing a cosmetic applicator, the method comprising:
providing an ultraviolet sterilizer that includes a housing with an interior within which at least one ultraviolet lamp is installed and enclosed, a power source connected to the at least one ultraviolet lamp and able to cause the at least one ultraviolet lamp to emit sterilizing ultraviolet light, and a hole in the housing through which a distal end of the cosmetic applicator can be inserted into the interior of the housing;
inserting the distal end of the cosmetic applicator through the hole and into the interior of the housing;
activating the power source, so as to cause the at least one ultraviolet lamp to emit sterilizing ultraviolet light;
allowing the sterilizing ultraviolet light to impinge on the distal end of the cosmetic applicator until an amount of ultraviolet light exposure is sufficient to sterilize the distal end of the cosmetic applicator; and
removing the distal end of the cosmetic applicator from the hole.
